(19) United States
(12) Patent Application Publication
(10) Pub. No.: US 2022/0415207 A1
(43) Pub. Date: Dec. 29, 2022

(54) METHOD AND TERMINAL FOR PROCESSING ELECTRONIC RECIPE, ELECTRONIC DEVICE

(71) Applicant: Shenzhen Chenbei Technology Co., Ltd., Shenzhen (CN)

(72) Inventors: Yujing MENG, Shenzhen (CN); Lei LUO, Shenzhen (CN)

(21) Appl. No.: 17/808,313

(22) Filed: Jun. 23, 2022

(30) Foreign Application Priority Data
Jun. 24, 2021 (CN) 202110705276.X

(51) Int. Cl. G09B 19/00; G06F 3/16; G06F 3/0488; G06F 3/04817; G06F 3/0483; G06F 3/0484; G16H 20/60

(52) U.S. Cl. CPC G09B 19/003; G06F 3/167; G06F 3/0488; G06F 3/04817; G06F 3/0483; G06F 3/0484; G09B 19/0092; G16H 20/60

(57) ABSTRACT

The application relates to a method and terminal for processing an electronic recipe, an electronic device. In the method, one presenting page is created for each recipe step of a target recipe, which facilitate a user to quickly locate a certain recipe step. Less content is shown on a single presenting page, which facilitates the user to quickly view operation content of a certain recipe step. Meanwhile, because the presenting page of each recipe step is configured with an interactive tool capable of being invoked by the user, and the interactive tool can guide the user to complete a corresponding recipe step, and as compared with a method in the prior art which requires manual and repeated memorizing or repeated viewing of a certain recipe step to complete cooking, the technical solutions of the application greatly improve cooking efficiency and user experience.

### REFERENCE TO RELATED APPLICATION

This is a non-provisional application which claims priority to a Chinese patent application having an application number of CN202110705276.X, and a filing date of Jun. 24, 2021, the entire contents of which is hereby incorporated by reference.

### TECHNICAL FIELD

The application relates to the field of electronic recipe technologies, and in particular, to a method and terminal for processing an electronic recipe, an electronic device.

### BACKGROUND

As people pay more attention to healthy diet, an increasing number of working people are more willing to cook at home. To pursue better color, aroma and taste, people usually rely on the guidance of electronic recipes in the cooking process.

In the prior art, electronic recipes usually include: web version recipes, video recipes, and picture recipes. The web version recipes are usually pure article-style recipes with a lot of text and with no picture or video guidance, are inconvenient to watch during cooking, and usually require a user to read complete recipe steps before cooking.

Although a video recipe facilitates viewing by the user, to ensure the user's viewing experience, the playback of a certain recipe step of the video recipe may be compressed. For example, a certain recipe step involves stewing for 30 minutes, but the step may be completed in a few seconds when the video recipe is played. In this case, if the user is still in the stewing stage while the video has skipped to the next stage after the stewing, after the user has completed the stewing step, the user still needs to watch the video again to complete the next recipe step.

With regard to a picture recipe, because all recipe steps are presented on one web page, a problem similar to that of web recipes occurs: it is inconvenient to watch the recipe during cooking, and the user usually needs to slide up and down to read the complete recipe steps before cooking. If the user forgets a certain recipe step during the cooking process, the user needs to interrupt the cooking and recheck the step, affecting user experience.

In conclusion, the web version recipes, the video recipes and the picture recipes present the recipe content in an excessively concentrated manner, it is difficult for the user to locate a certain recipe step, leading to low cooking efficiency and poor user experience.

### SUMMARY

In view of the above, an objective of the application is to provide a method and terminal for processing an electronic recipe, an electronic device, to solve the problem in the prior art that the content of an electronic recipe is presented in an excessively concentrated manner, which affects viewing by a user and leads to poor cooking efficiency.

According to a first aspect of embodiments of the application, a method for processing an electronic recipe applied to an electronic device is provided, including:

obtaining a source file of a target recipe, the source file including a plurality of recipe steps of the target recipe; and

creating one presenting page for each recipe step of the target recipe, where

the presenting page of each recipe step is configured with an interactive tool capable of being invoked by a user, and the interactive tool is configured to guide the user to complete a corresponding recipe step.

In a possible implementation solution, the interactive tool includes at least one of the following: a timer, a speech recognizer, a gesture recognizer, and a speech announcer.

In a possible implementation solution, under a condition that the interactive tool includes a timer, and any one recipe step is configured with timing, the presenting page of the recipe step is configured to start timing based on a user-triggered operation, and display countdown duration;

under a condition that the interactive tool includes a speech recognizer, the presenting page of the recipe step is configured to turn a page based on the user's page turning speech instruction;

under a condition that the interactive tool comprises a gesture recognizer, the presenting page of the recipe step is configured to turn a page based on page turning gesture instruction;

under a condition that the interactive tool includes a gesture recognizer, the presenting page of the recipe step is further configured to display a page turning gesture prompt icon under a condition that no user operation is detected within preset duration; and

under a condition that the interactive tool includes a speech announcer, the presenting page of the recipe step is configured to start speech broadcast of the recipe step based on a user instruction, and display a working state of the speech announcer.

In a possible implementation solution, it includes: under a condition that all recipe steps in the source file correspond to one parent file, prompting the user to specify a recipe step cutting node when uploading the source file, cutting the parent file into a plurality of subfiles based on the cutting node specified, wherein each subfile corresponds to a recipe step, and prompting the user to add operation content and a food ingredient description of each recipe step; and under a condition that each recipe step in the source file corresponds to a subfile, prompting the user to add operation content and a food ingredient description of each recipe step when uploading the source file.

In a possible implementation solution, the presenting page of each recipe step is further configured to: display the operation content, the food ingredient description and nutritional ingredient content calculated based on the food ingredient description of each recipe step, wherein the food ingredient description comprises food ingredient types and food ingredient weights; and under a condition that food ingredient types and/or food ingredient weights on any presenting page are modified by the user, calculate modified nutritional ingredient content in real time, and present a calculation result to the user.

In a possible implementation solution, the method further includes:

associating at least one cooking device with the target recipe, and establishing a list of cooking devices; obtaining recipe information of different cooking devices in the list of cooking devices; presenting recipe information of the user's default cooking device in the list of cooking devices, and the list of cooking devices on the detail page for the target recipe; and presenting recipe information of other cooking devices in the list of cooking devices on a switching page of the detail page for the target recipe, the switching page being entered by clicking on an icon of the other cooking devices in the list of cooking devices, where the detail page for the target recipe is provided with a touch button that controls the user's default cooking device to start cooking and a link for viewing the recipe information step by step; the switching page is provided with a touch button that controls a corresponding cooking device to start cooking and a link for viewing the recipe information step by step; and the link is associated with the presenting page of each recipe step.

In a possible implementation solution, the method further includes: creating a smart cooking parameter setup page after the presenting pages of all the recipe steps, where the smart cooking parameter setup page is used to provide the user with options for food ingredient types and food ingredient weights, and recommend an optimum cooking temperature and cooking duration based on food ingredient types and food ingredient weights selected by the user.

According to a second aspect of embodiments of the application, a terminal for processing an electronic recipe is provided, including:

an obtaining module, configured to obtain a source file of a target recipe, the source file including a plurality of recipe steps of the target recipe; and

a creation module, configured to, for each recipe step of the target recipe, create a presenting page, and configure an interactive tool capable of being invoked by a user, the interactive tool being configured to guide the user to complete a corresponding recipe step.

In other possible implementation solutions, the terminal for processing an electronic recipe is further configured to implement other possible implementation solutions according to the foregoing first aspect.

According to a third aspect of embodiments of the application, a method for displaying an electronic recipe applied to an electronic device is provided, including:

obtaining a source file of a target recipe, the source file including a plurality of recipe steps of the target recipe; and

displaying only one recipe step of the target recipe on one presenting page, where

the presenting page of each recipe step is configured with an interactive tool capable of being invoked by a user, and the interactive tool is configured to guide the user to complete a corresponding recipe step.

In a possible implementation solution, the interactive tool includes at least one of the following:

a timer, a speech recognizer, a gesture recognizer, and a speech announcer.

In a possible implementation solution, under a condition that the interactive tool includes a timer, the method further includes: under a condition that any one recipe step is timed during uploading, when the recipe step is displayed, starting timing based on a user-triggered operation, and displaying countdown duration on the presenting page of the recipe step;

under a condition that the interactive tool includes a speech recognizer, the method further includes: on the presenting page of the recipe step, turning a page based on the user's page turning speech instruction;

under a condition that the interactive tool comprises a gesture recognizer, the presenting page of the recipe step is configured to turn a page based on page turning gesture instruction;

under a condition that the interactive tool includes a gesture recognizer, the method further includes: on the presenting page of each recipe step, displaying a page turning gesture prompt icon under a condition that no user operation is detected within a preset duration; and

under a condition that the interactive tool includes a speech announcer, the method further includes: on the presenting page of each recipe step, starting speech broadcast of the recipe step based on a user instruction, and displaying a working state of the speech announcer.

In a possible implementation solution, the method further includes:

on the presenting page of each recipe step, displaying the operation content, the food ingredient description and nutritional ingredient content calculated based on the food ingredient description of each recipe step, wherein the food ingredient description includes food ingredient types and food ingredient weights; and

under a condition that food ingredient types and/or food ingredient weights on any presenting page are modified by the user, calculate modified nutritional ingredient content in real time, and present a calculation result to the user.

In a possible implementation solution, on the presenting page of each recipe step, the operation content and the food ingredient description of each recipe step are displayed in a font with a font size greater than a preset font size.

In a possible implementation solution, the method further includes:

displaying a detail page for the target recipe before presenting pages of all the recipe steps; associating at least one cooking device with the target recipe, and establishing a list of cooking devices; obtaining recipe information of different cooking devices in the list of cooking devices; presenting recipe information of the user's default cooking device in the list of cooking devices, and the list of cooking devices on the detail page for the target recipe; and presenting recipe information of other cooking devices in the list of cooking devices on a switching page of the detail page for the target recipe, the switching page being entered by clicking on an icon of the other cooking devices in the list of cooking devices, where the detail page for the target recipe is provided with a touch button that controls the user's default cooking device to start cooking and a link for viewing the recipe information step by step; the switching page is provided with a touch button that controls a corresponding cooking device to start cooking and a link for viewing the recipe information step by step; and the link is associated with the presenting page of each recipe step.

In a possible implementation solution, the method further includes: displaying a smart cooking parameter setup page after the presenting pages of all the recipe steps, where the smart cooking parameter setup page is used to provide the user with options for food ingredient types and food ingredient weights, and recommend an optimum cooking temperature and cooking duration based on food ingredient types and food ingredient weights selected by the user.

According to a fourth aspect of embodiments of the application, a terminal for displaying an electronic recipe is provided, including:

an obtaining module, configured to obtain a source file of a target recipe, the source file including a plurality of recipe steps of the target recipe; and

a presenting module, configured to present only one recipe step of the target recipe on one presenting page, where the presenting page of each recipe step is configured with an interactive tool capable of being invoked by a user, and the interactive tool is configured to guide the user to complete a corresponding recipe step.

In other possible implementation solutions, the terminal for processing an electronic recipe is further configured to implement other possible implementation solutions according to the foregoing third aspect.

According to a fifth aspect of embodiments of the application, an electronic device is provided, including:

a wireless communication module, a processor, and a memory, where program instructions are stored in the memory; and

the processor is configured to execute the program instructions stored in the memory, and perform the method according to the foregoing first aspect and the possible implementations thereof, or perform the method according to the foregoing third aspect and the possible implementations thereof.

According to a sixth aspect of embodiments of the application, a computer-readable storage medium is provided, where the computer-readable storage medium stores an erasable computer program; and

when the computer program runs on a computer device, the computer device is enabled to perform the method for processing an electronic recipe according to the foregoing first aspect and the possible implementations thereof, and/or perform the method for displaying an electronic recipe according to the foregoing third aspect and the possible implementations thereof.

The technical solutions according to the application may achieve the following beneficial effects:

One presenting page is created for each recipe step of a target recipe, which facilitate a user to quickly locate a certain recipe step. Less content is shown on a single presenting page, which facilitates the user to quickly view operation content of a certain recipe step. Meanwhile, because the presenting page of each recipe step is configured with an interactive tool capable of being invoked by the user, and the interactive tool can guide the user to complete a corresponding recipe step, compared with a method in the prior art which requires manual and repeated memorizing or repeated viewing of a certain recipe step to complete the cooking, the technical solutions of the application greatly improve cooking efficiency and user experience.

It should be understood that the foregoing general description and the following detailed description are only examples and are explanatory, and cannot limit the application.

### BRIEF DESCRIPTION OF THE DRAWINGS

The accompanying drawings herein are incorporated into the specification and constitute a part of the specification, show embodiments that conform to the application, and are used together with the specification to explain the principle of the application.

FIG. 1 is a flowchart showing a method for processing an electronic recipe according to an exemplary embodiment;

FIG. 2 is a schematic diagram showing a presenting page of a recipe step according to an exemplary embodiment when a food ingredient description is modified;

FIG. 3 is a schematic diagram showing a presenting page of a recipe step according to an exemplary embodiment when a timer is configured;

FIG. 4 is a schematic diagram showing a presenting page of a recipe step according to an exemplary embodiment when a gesture recognizer is configured;

FIG. 5 is a schematic diagram showing a presenting page of a recipe step according to an exemplary embodiment when a speech announcer is configured;

FIG. 6 is a schematic diagram showing a detail page for the target recipe according to an exemplary embodiment;

FIG. 7 is a flowchart showing a method for processing an electronic recipe according to another exemplary embodiment;

FIG. 8 is a schematic block diagram showing a terminal for processing an electronic recipe according to an exemplary embodiment;

FIG. 9 is a flowchart showing a method for displaying an electronic recipe according to an exemplary embodiment; and

FIG. 10 is a schematic block diagram showing a terminal for displaying an electronic recipe according to an exemplary embodiment.

### DETAILED DESCRIPTION OF THE PREFERRED EMBODIMENT

Example embodiments are described in detail herein, and examples thereof are shown in the accompanying drawings. When the following description refers to the accompanying drawings, unless otherwise indicated, the same numbers in different accompanying drawings indicate the same or similar elements. The implementations described in the following example examples do not represent all implementations consistent with the application. On the contrary, the implementations are merely examples of apparatuses and methods consistent with some aspects of the application and detailed in the appended claims.

As described in the foregoing background art, in the prior art, the content of an electronic recipe is presented in an excessively concentrated manner, which affects viewing by a user and leads to poor cooking efficiency.

To solve the technical problems, the application provides three technical solutions, which are specifically as follows:

The first technical solution: a method and terminal for processing an electronic recipe, an electronic device, and a computer-readable storage medium; and this technical solution solves the foregoing technical problem by improving the method for processing an electronic recipe.

The second technical solution: a method and terminal for displaying an electronic recipe, an electronic device, and computer-readable storage medium; and this technical solution solves the foregoing technical problem by improving the method for displaying an electronic recipe.

The implementation of each technical solution is described with reference to specific embodiments.

It should be noted that the “target recipe” described in all embodiments of the application is the name of the dish that the user is going to cook, and detail page for the target recipe is recipe information corresponding to dishes that the user prepares to cook. For example, if the user is preparing to cook scrambled eggs with tomatoes, the detail page for the target recipe is recipe information required for the scrambled eggs with tomatoes. The recipe information includes at least: operation content, a food ingredient description, a cooking temperature, and cooking duration corresponding to each operation step.

The implementation of the first technical solution is described in detail with reference to Embodiment I to Embodiment IV.

### Embodiment I

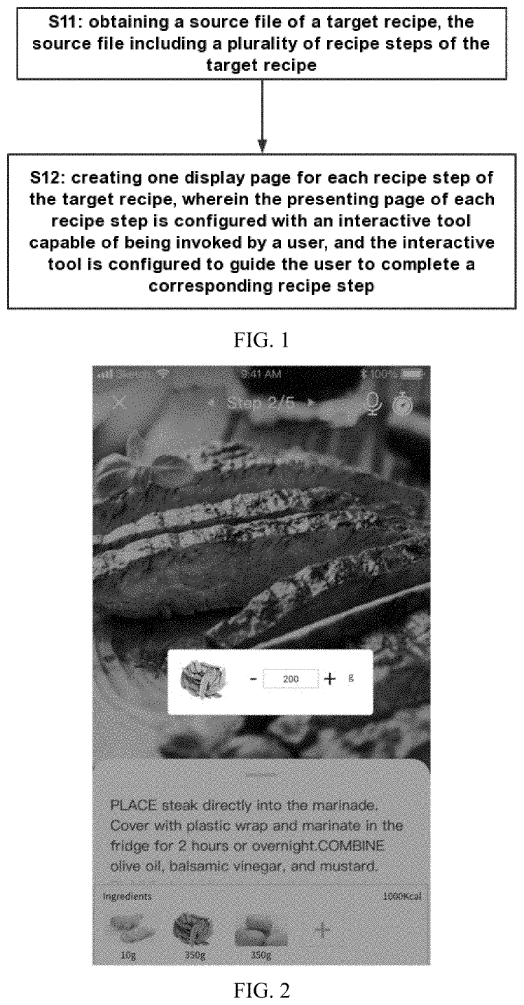

FIG. 1 is a flowchart showing a method for processing an electronic recipe according to an exemplary embodiment. As shown in FIG. 1, the method includes:

step S11: obtaining a source file of a target recipe, the source file including a plurality of recipe steps of the target recipe; and

step S12: creating one presenting page for each recipe step of the target recipe, wherein the presenting page of each recipe step is configured with an interactive tool capable of being invoked by a user, and the interactive tool is configure is provided with d to guide the user to complete a corresponding recipe step.

As can be understood that in the technical solution in this embodiment, one presenting page is created for each recipe step of a target recipe, which facilitate a user to quickly locate a certain recipe step. Less content is shown on a single presenting page, which facilitates the user to quickly view operation content of a certain recipe step. In addition, because the presenting page of each recipe step is configured with an interactive tool capable of being invoked by the user, and the interactive tool can guide the user to complete a corresponding recipe step, compared with a method in the prior art which requires manual and repeated memorizing or repeated viewing of a certain recipe step to complete the cooking, the technical solution in this embodiment greatly improves cooking efficiency and user experience.

It should be noted that during specific operation, the technical solution in this embodiment needs to be loaded and run in an electronic device, or loaded and run in a user APP in the electronic device. The electronic device includes: a server-side electronic device and a user-side electronic device, including but not limited to: a mobile phone, a computer, a notebook computer, a tablet computer, an intelligent wearable device, or the like. The tablet computer may be a household handheld tablet computer, or a tablet computer embedded in a kitchen device, for example, a tablet computer embedded in a range hood, a tablet computer embedded in an oven, or a tablet computer embedded in a refrigerator.

It should be noted that if the technical solution in this embodiment runs in a user APP loaded in an electronic device, if a permission of the user APP is set as that the user only has a permission to process an electronic recipe, then in this case, the user can only process an electronic recipe and view the electronic recipe processed by the user.

If the permission of the user APP is set as that the user can have a permission to process an electronic recipe and a viewing permission, in this case, the user can not only process an electronic recipe, but also view electronic recipes shared by others.

Obtaining a source file of a target recipe in step S11 may be implemented in the following ways in specific practice:

receiving a source file of a target recipe uploaded by the user through a server-side electronic device, and/or,

receiving a source file of a target recipe uploaded by the user through a user-side electronic device, where

the source file is at least one of the following forms:

a video, a picture, and text.

As described above, the technical solution in this embodiment may be loaded on a server-side electronic device and a user-side electronic device for operation. If the technical solution in this embodiment is loaded on a server-side electronic device, a background manager may be equipped on a server side, so that the background manager uploads source files of various electronic recipes. If the technical solution in this embodiment is loaded on a user-side electronic device, users of the user-side electronic device may be allowed to share source files of electronic recipes.

In specific practice, regardless of the form in which the source file of the electronic recipe is obtained, the obtained source file of the electronic recipe may be saved in a database on the server side. If the server side needs to obtain the source file of the target recipe, the source file is directly retrieved from the database; if the user side needs to obtain the source file of the target recipe, the user side may send a request to the server side, and the server side will match against the source file of the target recipe.

As can be understood that regardless of the form in which the user uploads the source file of the target recipe, the source file may be one of the following two forms:

1. All recipe steps in the source file correspond to one parent file. Taking a target recipe of scrambled eggs with tomatoes as an example, the user may upload at least one of the following files: a complete video of the scrambled eggs with tomatoes, a complete picture explanation of the scrambled eggs with tomatoes, and a document description of the scrambled eggs with tomatoes.

2. Each recipe step in the source file corresponds to one subfile. Taking a target recipe of scrambled eggs with tomatoes as an example, the user may upload at least one of the following files: a step-by-step video of the scrambled eggs with tomatoes, each step video corresponding to one subfile; and/or, a step-by-step picture explanation of the scrambled eggs with tomatoes, each step picture corresponding to one subfile; and/or, a step-by-step document description of the scrambled eggs with tomatoes, each step description corresponding to one subfile.

In specific practice, the foregoing two source file formats 1 and 2 may be processed by the technical solution in this embodiment, specifically:

under a condition that all recipe steps in the source file correspond to one parent file, prompting the user to specify a recipe step cutting node when uploading the source file, cutting the parent file into a plurality of subfiles based on the cutting node specified, wherein each subfile corresponds to a recipe step, and prompting the user to add operation content and a food ingredient description of each recipe step; and

under a condition that each recipe step in the source file corresponds to a subfile, prompting the user to add operation content and a food ingredient description of each recipe step when uploading the source file.

As can be understood that as people pay more attention to health, an increasing number people are beginning to pay attention to the balance between the taste of dishes and health. Some people even need to strictly control the nutritional ingredient content of each dish to lose weight and shape their bodies. Because a user of an electronic recipe may need to understand the nutritional ingredient content of each dish, in specific practice, the following solution may be adopted:

displaying, on a presenting page of a corresponding recipe step, the operation content, the food ingredient description and nutritional ingredient content calculated based on the food ingredient description of each recipe step, where

the food ingredient description includes: food ingredient types and food ingredient weights.

In specific practice, on the presenting page of each recipe step, the operation content and the food ingredient description corresponding to each recipe step are displayed in a font with a font size greater than a preset font size.

The preset font size may be set based on user needs, for example, may be set to Sihao Kaiti or Sanhao Songti. The font display with a front size greater than the preset font size can help users with poor eyesight, and can also help ordinary users. This is because after the font is enlarged, the user can see the font more clearly, which facilitates cooking.

In addition, during the cooking process, the user may adjust food ingredient types and food ingredient weights of a certain recipe step based on actual needs. When food ingredient types and food ingredient weights of a certain recipe step are modified by the user, the final nutritional ingredient content (for example, the content of calcium, iron, zinc, and protein) of an entire dish will also be changed. Therefore, in specific practice, the following solution may be used:

under a condition that food ingredient types and/or food ingredient weights on any presenting page are modified by the user (referring to FIG. 2), calculating modified nutritional ingredient content in real time, and presenting a calculation result to the user.

This allows the user to learn about (for example, no matter whether the user is cooking, the user can learn about) the updated nutritional ingredient content in real time, and then control the increase or decrease of subsequent food ingredients, so as to control the nutritional ingredient content of the entire dish and meet the user's requirements for personalized cooking.

The interactive tool described in step S12 includes at least one of the following:

a timer, a speech recognizer, a gesture recognizer, and a speech announcer.

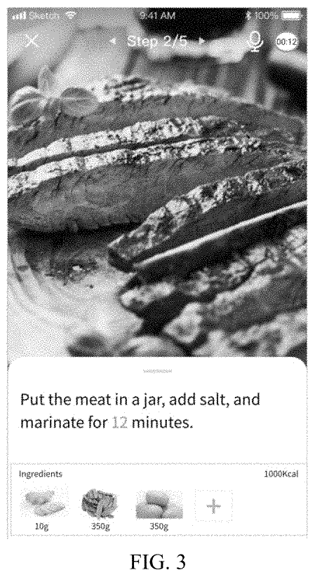

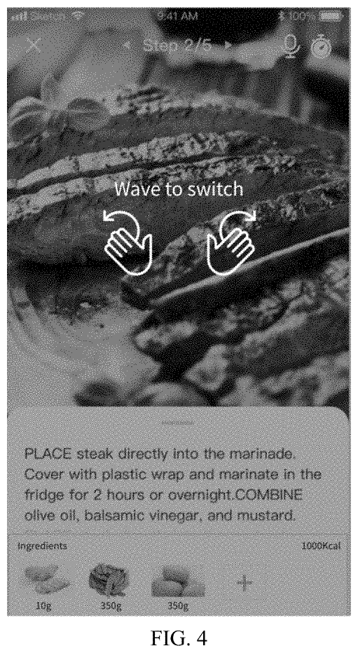

Referring to FIG. 3, in specific practice, under a condition that the interactive tool includes a timer, and any one recipe step is configured with timing, the presenting page of the recipe step is configured to start timing based on a user-triggered operation, and display countdown duration.

It should be noted that if any one recipe step is configured with timing, the recipe step may be configured with timing during uploading, or during displaying.

If timing is configured during uploading, when the recipe step is displayed, the user does not need to set the timing through other applications, just trigger the timer to start. If the recipe step is not configured with timing during uploading, when the recipe step is displayed, the user needs to use another application set the timing, and then triggers the timer for starting the timing.

To achieve a better experience effect, preferably, in this embodiment, “any one recipe step is configured with timing during uploading”.

Certainly, regardless of the configuration method, this is only an example. In this embodiment, the configuration of a timing function for any one recipe step under a specific situation is not limited, for example, the configuration is modified again after the recipe is uploaded.

Regardless of the timing, as long as the timing is required in the cooking process, the timer can be started for timing. If the timing is set when a background user uploads the recipe step, a foreground user does not need to interrupt the cooking to set the timing when using the electronic recipe, and can start set timing by a trigger operation (for example, manually clicking a timer icon on the current presenting page to start the timing, or starting the timing by using a preset speech instruction), thereby saving time and labor, making user experience good, and making satisfaction high.

As can be understood that adding a timing function to a presenting page of a specified recipe step can help the user to time. During the cooking process, the user uses the timing function generally in two situations: operation timing and cooking timing. The operation timing refers to various preparation timings, for example, various operation timings required by the user, such as marinating beef for 12 minutes and soaking vermicelli in hot water for 10 hours. The cooking timing refers to duration of cooking by using a cooking device required in the current recipe step if the target recipe is associated with the cooking device, or timing duration that the user needs to wait in the cooking step. For example, if a target recipe is roasted sweet potatoes, a cooking device associated with the target recipe is an oven, and the current recipe step is “preheating the oven at 220° C., and baking at a middle layer for 20 minutes”, then the cooking time is the 20 minutes during which the oven needs to bake. In addition, if the user needs to check a cooking situation after 15 minutes, the user may set a timer for 15 minutes on the presenting page of the recipe step.

In specific practice, under a condition that the interactive tool includes a speech recognizer and/or a gesture recognizer, the presenting page of each recipe step is configured to turn a page based on the user's page turning speech instruction and/or page turning gesture instruction. Under a condition that the interactive tool comprises a speech recognizer, the presenting page of the recipe step is configured to turn a page based on the user's page turning speech instruction; and under a condition that the interactive tool comprises a gesture recognizer, the presenting page of the recipe step is configured to turn a page based on the page turning gesture instruction.

It should be noted that, in specific practice, the page turning speech instruction and the page turning gesture instruction may be flexibly set based on user needs. For example, the page turning speech instruction is preset as “previous page” and “next page” to implement page up and page down; and the page turning gesture instruction is preset as “left wave” and “right wave” to implement page up and page down.

As can be understood that the user turns a page by using the page turning speech instruction and/or the page turning gesture instruction without directly touching the electronic device, which frees the user's hands. Meanwhile, the user can complete the page turning of the target recipe without leaving a cooking site, enabling the user to pay more attention to cooking, facilitating recipe viewing by the user while further improving cooking efficiency.

In specific practice, referring to FIG. 4, under a condition that the interactive tool includes a gesture recognizer, the presenting page of each recipe step is further configured to display a page turning gesture prompt icon under a condition that no user operation is detected within a preset duration.

It should be noted that the preset duration is set based on user needs, for example, set to 30 seconds. The user operation refers to a touch operation and a speech control operation performed by the user on the electronic recipe, including but not limited to: page up, page down, pause, and a timing trigger operation.

The display of a page turning gesture prompt icon may be a suspended display of the page turning gesture prompt icon on the presenting page of the current recipe step.

As can be understood that if no user operation is detected within the preset duration, the system defaults that the user is not clear about how to implement non-contact page turning, and the page turning gesture prompt icon is displayed, which can help the user to correctly use the preset page turning gesture instruction and improve cooking efficiency.

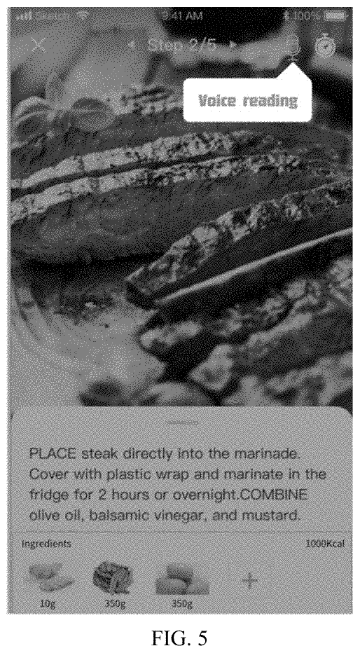

In specific practice, referring to FIG. 5, under a condition that the interactive tool includes a speech announcer, the presenting page of the recipe step is configured to start speech broadcast of the recipe step based on a user instruction, and display a working state of the speech announcer.

As can be understood that during the cooking process, the user's eyes are liberated through the speech broadcast of the recipe step, and the user is listening while cooking, so that the user can focus more on the cooking of a dish, which improves cooking efficiency and user experience.

Meanwhile, because one presenting page displays only one recipe step, the speech broadcast content of the presenting page of each recipe step is less, the content of a single broadcast is more easily absorbed by the user, and the user experience is better.

Because the speech announcer starts the speech broadcast of the recipe step based on the user instruction, the speech announcer can provide repeated broadcast for many times, and prevent the user from missing key information due to a noisy cooking environment, thereby achieving a better effect of absorption by the user and better experience, and also helping users with poor eyesight.

As can be understood that when different cooking devices are used to cook the same dish, corresponding recipe information may be different. For example, if a target recipe is steamed egg custard, using a wok to make the steamed egg custard, using a steamer to make the steamed egg custard, using a microwave oven to make the steamed egg custard and using a rice cooker to make the steamed egg custard require different water consumption, cooking duration, and cooking temperatures.

For the same target recipe, considering that the recipe information corresponding to different cooking devices may be different, the following solution may be adopted:

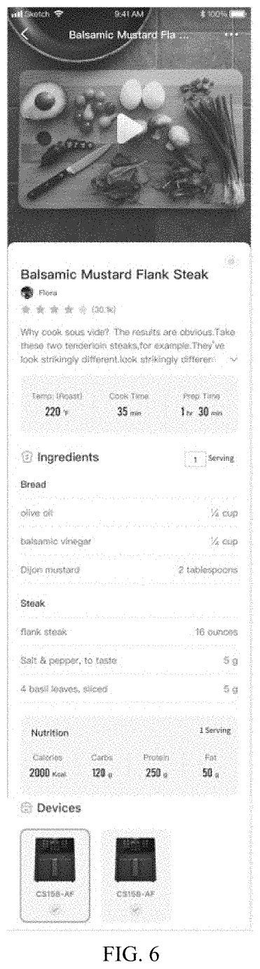

1. referring to FIG. 6, creating a detail page for the target recipe before presenting pages of all the recipe steps.

2. associating at least one cooking device with the target recipe, and establishing a list of cooking devices.

It should be noted that the “cooking device” refers to a cooking device that can be used when a target recipe is cooked in specific practice. For example, if the target recipe is steamed egg custard, the cooking device includes: a wok, a steamer, a microwave oven, a rice cooker, or the like; and for another example, if the target recipe is stewed ribs with lotus root, a cooking device includes: a casserole, a wok, a pressure cooker, a rice cooker, or the like.

The “associating at least one cooking device” refers to selecting one or more of the foregoing “cooking devices” for association. Cooking devices in the “cooking device list” only refers to cooking devices that are associated with the target recipe. In an optional solution, at least one cooking device associated with the target recipe are cooking devices that establish a binding relationship with the software/APP; and it is more convenient to provide a cooking recipe for devices owned by the user.

For example, if a target recipe is steamed egg custard, the cooking device includes: a wok, a steamer, a microwave oven, a rice cooker, or the like; and if the user associates the target recipe steamed egg custard with the steamer and the rice cooker among the cooking devices, then only the steamer and the rice cooker are in the list of cooking devices.

In specific practice, “associating at least one cooking device with the target recipe” may be implemented by the following solution (the following methods are only examples, and this embodiment is not limited to only the following implementations):

Solution I: starting a communication module (for example, a Wi-Fi module, a Bluetooth module, a 4G communications module, or the like) of a cooking device, and then starting a communication module of an electronic device running the method for processing an electronic recipe in this embodiment; and after the electronic device searches for a communication signal of the cooking device within a preset space range, establishing a communication link with the cooking device based on the user's authorization, thereby associating the cooking device with the target recipe.

Solution II: loading an APP that controls the working of the cooking device into the electronic device, and also loading the method for processing an electronic recipe in this embodiment into the electronic device. In the case of obtaining the user's authorization, the electronic device reads device parameters and control parameters in the APP of the cooking device that has been loaded in the electronic device, and loads the obtained device parameters and control parameters into the electronic recipe for invoking by the user, thereby associating the cooking device with the target recipe.

Solution III: in the case of obtaining the user's authorization, indirectly associating the cooking device with the target recipe by using the electronic recipe as a main application, and using the APP of the cooking device as a secondary application.

In conclusion, to associate the cooking device with the target recipe, on the one hand, the cooking device needs to have a remote control function, and is a smart cooking device, on the other hand, the user's authorization is further required, and the cooking device that needs to be associated is selected from smart cooking devices based on the user's authorization.

3. Recipe information of different cooking devices (including different models of cooking devices and different types of cooking devices) in the list of cooking devices is obtained.

As can be understood that even if for the same dish, when cooking devices are different, the recipe information may be different. Taking the target recipe being steamed egg custard as an example, the water consumption, steps and cooking duration required for cooking with a steamer are different from those with a rice cooker.

Then, how the “obtaining recipe information of different cooking devices in the list of cooking devices” is implemented?

In specific practice, the system may require the user to select a cooking device used by an electronic recipe when uploading the electronic recipe, so that each electronic recipe created corresponds to one cooking device.

For example, taking the target recipe being steamed egg custard as an example, an electronic recipe uploaded by user A is implemented by cooking in a steamer, and then the system saves a piece of information: target recipe-steamed egg custard-steamer-recipe information; and an electronic recipe uploaded by user B is implemented by cooking in a rice cooker, and the system saves another piece of information: target recipe-steamed egg custard-rice cooker-recipe information, . . . , and so on.

Based on this, after the user associates the cooking device with the target recipe, the system can match against the corresponding recipe information based on the cooking device.

It should be noted that the recipe information includes at least: operation content, a food ingredient description, a cooking temperature, and cooking duration corresponding to each recipe step.

4. Recipe information of the user's default cooking device in the list of cooking devices, and the list of cooking devices are displayed on the detail page for the target recipe.

5. Recipe information of other cooking devices in the list of cooking devices is displayed on a switching page of the detail page for the target recipe, and the switching page is entered by clicking on an icon of the other cooking devices in the list of cooking devices.

Still taking the target recipe being steamed egg custard as an example, if a steamer and a rice cooker are in the list of cooking devices, two sets of recipe information are provided under the target recipe. One is the recipe information when the steaming egg custard is made with the steamer, and the other is the recipe information when the steaming egg custard is made in a rice cooker.

If both sets of recipe information are displayed on the target detail page, a lot of content are presented on the target detail page, which reduces the user's perception and experience. Therefore, the technical solution in this embodiment is that one page displays one set of recipe information of the target recipe, and one set of recipe information corresponds to one cooking device. In this way, the information quantity of each page can be reduced, and the page content is more concise and easy for the user to understand and absorb.

6. The detail page for the target recipe is provided with a touch button that controls the user's default cooking device to start cooking and a link for viewing the recipe information step by step; the switching page is provided with a touch button that controls a corresponding cooking device to start cooking and a link for viewing the recipe information step by step; and the link is associated with the presenting page of each recipe step.

As can be understood that, referring to FIG. 6, the detail page for the target recipe or a switching page thereof is equivalent to an overview page. The detail page for the target recipe corresponds to the user's default cooking device, and the page displays recipe information such as a complete video, a text description, cooking temperatures, cooking duration, food ingredient types and food ingredient weights, and nutritional ingredient content of the target recipe. Due to the more content of the text description part, only the foregoing part of the text description is displayed. The following content is displayed in a collapsed manner. The user can view the complete recipe step description by clicking a collapse button on the page. This page is provided with a link for viewing the recipe information step by step. Switching pages correspond to other cooking devices, and one switching page corresponds to one cooking device. Because the switching page and the detail page for the target recipe have the same layout and display content, details are no longer described herein.

As can be understood that because one cooking device corresponds to one set of recipe information, and one set of recipe information corresponds to the presenting pages of a plurality of recipe steps, the technical solution in this embodiment meets requirements of some user groups for viewing complete recipe information of the target recipe, also meets requirements of some user groups for viewing the recipe information step by step, and further meets requirements of some user groups for viewing the recipe information based on cooking device types and models. The user group compatibility is high and user experience is good.

In addition, through the arrangement of the touch button for starting the cooking, the combination of the target recipe and cooking practice is implemented. The user does not need to manually start the cooking device, or go to an APP of the cooking device to control the cooking device. The technical solution in this embodiment integrates three functions, creation of the electronic recipe, viewing, and cooking, and implements a full set of solutions from back-end production to front-end guided cooking and automated cooking, which improves user experience and increases user stickiness.

As can be understood that even if for the same cooking device, when food ingredient types and food ingredient weights are different, cooking methods of the target recipes may be different.

Taking a target recipe being steamed egg custard as an example, the cooking device is a steamer. When three eggs and eight eggs are cooked, to achieve the intended cooking effect (for example, tender and smooth), cooking temperatures used and cooking duration are definitely different. Therefore, in specific practice, the following solution may be used to meet the user's requirements for personalized recipe information customization, specifically:

creating a smart cooking parameter setup page after the presenting pages of all the recipe steps, where

the smart cooking parameter setup page is used to provide the user with options for food ingredient types and food ingredient weights, and recommend an optimum cooking temperature and cooking duration based on food ingredient types and food ingredient weights selected by the user.

In specific practice, cooking experiments can be used to record a corresponding relationship between food ingredient types and food ingredient weights, and different cooking temperatures and cooking duration when the preset cooking effects are achieved;

a cooking curve is drawn and saved based on the corresponding relationship; and

the recommend an optimum cooking temperature and cooking duration specifically includes:

recommending an optimum cooking temperature based on a pre-saved cooking curve.

It can be learned from the foregoing description that in specific practice, a plurality of implementations of the method for processing an electronic recipe are provided, and each implementation can solve one technical problem, and is not limited to the one shown in FIG. 1.

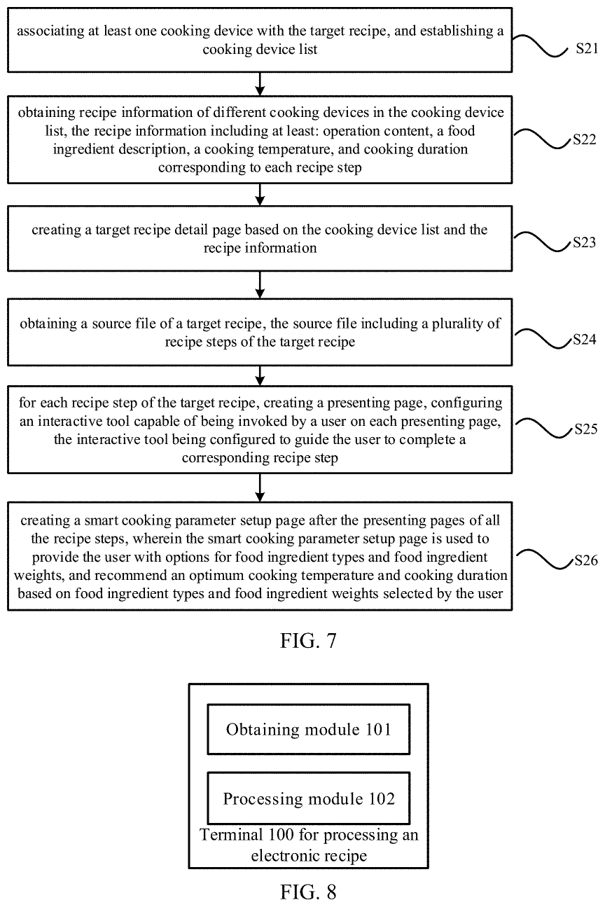

As shown in FIG. 7, FIG. 7 is a flowchart showing a method for processing an electronic recipe according to another exemplary embodiment. As shown in FIG. 7, the method includes:

step S21: associating at least one cooking device with a target recipe, and establishing a list of cooking devices;

step S22: obtaining recipe information of different cooking devices in the list of cooking devices, the recipe information including at least: operation content, a food ingredient description, a cooking temperature, and cooking duration corresponding to each recipe step;

step S23: creating a detail page for the target recipe based on the list of cooking devices and the recipe information;

step S24: obtaining a source file of the target recipe, the source file including a plurality of recipe steps of the target recipe;

step S25: for each recipe step of the target recipe, creating a presenting page, and configuring an interactive tool capable of being invoked by a user, the interactive tool being configured to guide the user to complete a corresponding recipe step; and

step S26: creating a smart cooking parameter setup page after the presenting pages of all the recipe steps, where

the smart cooking parameter setup page is used to provide the user with options for food ingredient types and food ingredient weights, and recommend an optimum cooking temperature and cooking duration based on food ingredient types and food ingredient weights selected by the user.

It should be noted that, because the implementations and beneficial effects of the foregoing steps have been described in detail above, the details are not described in this embodiment.

As can be understood that the technical solution in this embodiment provides a complete set of solutions including the construction of the detail page for the target recipe, the construction of the presenting page of each recipe step, and the construction of the smart cooking parameter setup page, which not only solves the problem in the prior art that the content of an electronic recipe is presented in an excessively concentrated manner, which affects viewing by the user and leads to poor cooking efficiency, but also meets various user's requirements for viewing personalized recipe information and customizing recipe information. The user experience is good and the satisfaction is high.

### Embodiment II

FIG. 8 is a schematic block diagram showing a terminal 100 for processing an electronic recipe according to an exemplary embodiment. As shown in FIG. 8, the terminal 100 includes:

an obtaining module 101, configured to obtain a source file of a target recipe, the source file including a plurality of recipe steps of the target recipe; and

a creation module 102, configured to, for each recipe step of the target recipe, create a presenting page, and configure an interactive tool capable of being invoked by a user, the interactive tool being configured to guide the user to complete a corresponding recipe step.

The obtaining module 101 is configured to obtain a source file of a target recipe, which may be implemented in the following ways in specific practice:

receiving a source file of a target recipe uploaded by the user through a server, and/or,

receiving a source file of a target recipe uploaded by the user through a user side, where

the source file is at least one of the following forms:

a video, a picture, and text.

The interactive tool configured by the creation module 102 for the presenting page of each recipe step includes at least one of the following:

a timer, a speech recognizer, a gesture recognizer, and a speech announcer.

It should be noted that, because for the implementations and beneficial effects of the foregoing modules, reference may be made to the detailed description of the corresponding steps in the foregoing embodiments, the details are not described in this embodiment.

### Embodiment III

An electronic device shown according to an exemplary embodiment is provided. The electronic device includes:

a wireless communication module, a processor, and a memory, where program instructions are stored in the memory; and

the processor is configured to execute the program instructions stored in the memory, and perform the foregoing method for processing an electronic recipe.

It should be noted that the electronic device includes, but is not limited to: an intelligent terminal (for example, a mobile phone, a tablet computer, or a smartwatch) and a computer device.

The wireless communication module includes, but is not limited to: a WCDMA, a GSM, a CDMA and/or an LTE communication module. Wireless methods mainly include short-range wireless communication technologies such as an infrared technology, a ZigBee technology, a Bluetooth technology, a low-frequency radio technology, and a 2.4G radio frequency technology.

The processor includes, but is not limited to: a CPU, a single-chip microcomputer, a PLC, an FPGA controller, and the like.

The memory may include a computer system readable medium in the form of a volatile memory, such as random access memory (RAM) and/or a cache; or include another removable/non-removable, volatile/nonvolatile computer system storage medium. The memory may include at least one program product, and the program product has a group of (for example, at least one) program modules, and these program modules are configured to perform functions of the embodiments of the application.

As can be understood that in the technical solution in this embodiment, one presenting page is created for each recipe step of a target recipe, which facilitate a user to quickly locate a certain recipe step. Less content is shown on a single presenting page, which facilitates the user to quickly view operation content of a certain recipe step. In addition, because the presenting page of each recipe step is configured with an interactive tool capable of being invoked by the user, and the interactive tool can guide the user to complete a corresponding recipe step, compared with a method in the prior art which requires manual and repeated memorizing or repeated viewing of a certain recipe step to complete the cooking, the technical solution in this embodiment greatly improves cooking efficiency and user experience.

### Embodiment IV

A computer-readable storage medium shown according to an exemplary embodiment is provided, where the computer-readable storage medium stores an erasable computer program; and

when the computer program runs on a computer device, the computer device is enabled to perform the foregoing method for processing an electronic recipe.

The computer-readable storage medium disclosed in this embodiment includes, but is not limited to: an electrical, magnetic, optical, electromagnetic, infrared, or semiconductor system, apparatus or device, or a combination of arbitrary ones of the above. More specific examples (a non-exhaustive list) of computer-readable storage media include: an electrical connector with one or more wires, a portable computer disk, a hard disk, a random access memory (RAM), a read-only memory (ROM), an erasable programmable read-only memory (EPROM or flash memory), an optical fiber, a portable compact disk read-only memory (CD-ROM), an optical storage device, a magnetic storage device, or any suitable combination of the above. In the application, the computer-readable storage medium may be any tangible medium that contains or stores a program, and the program may be used by or in combination with an instruction execution system, apparatus or device.

As can be understood that in the technical solution in this embodiment, one presenting page is created for each recipe step of a target recipe, which facilitate a user to quickly locate a certain recipe step. Less content is shown on a single presenting page, which facilitates the user to quickly view operation content of a certain recipe step. In addition, because the presenting page of each recipe step is configured with an interactive tool capable of being invoked by the user, and the interactive tool can guide the user to complete a corresponding recipe step, compared with a method in the prior art which requires manual and repeated memorizing or repeated viewing of a certain recipe step to complete the cooking, the technical solution in this embodiment greatly improves cooking efficiency and user experience.

The implementation of the second technical solution is described in detail with reference to Embodiment V to Embodiment VIII.

### Embodiment V

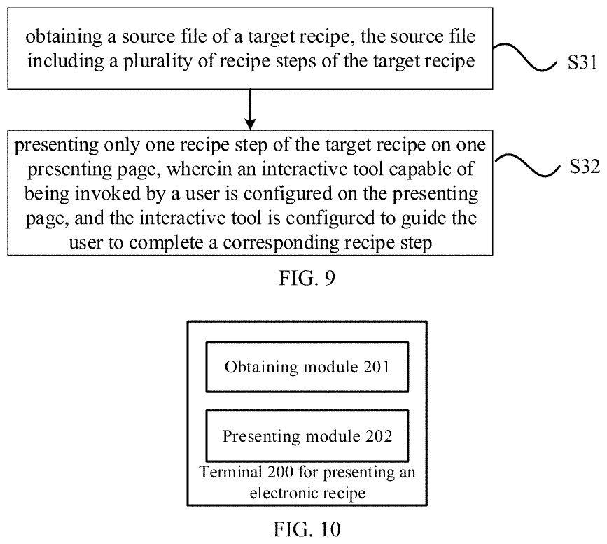

FIG. 9 is a flowchart showing a method for displaying an electronic recipe according to an exemplary embodiment. As shown in FIG. 9, the method includes:

step S31: obtaining a source file of a target recipe, the source file including a plurality of recipe steps of the target recipe; and

step S32: displaying only one recipe step of the target recipe on one presenting page, where

the presenting page of each recipe step is configured with an interactive tool capable of being invoked by a user, and the interactive tool is configured to guide the user to complete a corresponding recipe step.

As can be understood that in the technical solution in this embodiment, only one recipe step of the target recipe is displayed on one presenting page, which facilitate a user to quickly locate a certain recipe step. Less content is shown on a single presenting page, which facilitates the user to quickly view operation content of a certain recipe step. Meanwhile, because the presenting page of each recipe step is configured with an interactive tool capable of being invoked by the user, and the interactive tool can guide the user to complete a corresponding recipe step, compared with a method in the prior art which requires manual and repeated memorizing or repeated viewing of a certain recipe step to complete the cooking, the technical solution in this embodiment greatly improves cooking efficiency and user experience.

It should be noted that during specific operation, the technical solution in this embodiment needs to be loaded and run in an electronic device, or loaded and run in a user APP in the electronic device. The electronic device includes: a server-side electronic device and a user-side electronic device, including but not limited to: a mobile phone, a computer, a notebook computer, a tablet computer, an intelligent wearable device, or the like. The tablet computer may be a household handheld tablet computer, or a tablet computer embedded in a kitchen device, for example, a tablet computer embedded in a range hood, a tablet computer embedded in an oven, or a tablet computer embedded in a refrigerator.

It should be noted that if the technical solution in this embodiment runs in a user APP loaded in an electronic device, if a permission of the user APP is set as that the user only has a permission to view an electronic recipe, then in this case, the user can only view electronic recipes shared by others.

If the permission of the user APP is set as that the user can have a permission to process an electronic recipe and a viewing permission, in this case, the user can not only process an electronic recipe, but also view electronic recipes shared by others.

As can be understood that as people pay more attention to health, an increasing number people are beginning to pay attention to the balance between the taste of dishes and health. Some people even need to strictly control the nutritional ingredient content of each dish to lose weight and shape their bodies. Because a user of an electronic recipe may need to understand the nutritional ingredient content of each dish, in specific practice, the following solution may be adopted:

on the presenting page of each recipe step, displaying the operation content, the food ingredient description and nutritional ingredient content calculated based on the food ingredient description of each recipe step, where

the food ingredient description includes: food ingredient types and food ingredient weights.

In addition, during the cooking process, the user may adjust food ingredient types and food ingredient weights of a certain recipe step based on actual needs. When food ingredient types and food ingredient weights of a certain recipe step are modified by the user, the final nutritional ingredient content (for example, the content of calcium, iron, zinc, and protein) of an entire dish will also be changed. Therefore, in specific practice, the following solution may be used:

under a condition that food ingredient types and/or food ingredient weights on any presenting page are modified by the user (referring to FIG. 2), calculating modified nutritional ingredient content in real time, and presenting a calculation result to the user.

This allows the user to learn about the updated nutritional ingredient content in real time, and then control the increase or decrease of subsequent food ingredients, so as to control the nutritional ingredient content of the entire dish and meet the user's requirements for personalized cooking.

In specific practice, to help the user with poor eyesight, on the presenting page of each recipe step, the operation content and the food ingredient description of each recipe step are displayed in a font with a font size greater than a preset font size.

The preset font size may be set based on user needs.

The interactive tool described in step S32 includes at least one of the following:

a timer, a speech recognizer, a gesture recognizer, and a speech announcer.

It should be noted that the method for displaying an electronic recipe in this embodiment is implemented based on the method for processing an electronic recipe in Embodiment I. On the basis of the method for processing an electronic recipe in Embodiment I, how to display the electronic recipe can be expected. Therefore, for the specific implementations and beneficial effects of step S31 and step S32 in this embodiment, reference may be made to the related description of Embodiment I. Details are not described in this embodiment.

### Embodiment VI

FIG. 10 is a schematic block diagram showing a terminal 200 for presenting an electronic recipe according to an exemplary embodiment. As shown in FIG. 10, the terminal 200 includes:

an obtaining module 201, configured to obtain a source file of a target recipe, the source file including a plurality of recipe steps of the target recipe; and

a presenting module 202, configured to present only one recipe step of the target recipe on one presenting page, where the presenting page of each recipe step is configured with an interactive tool capable of being invoked by a user, and the interactive tool is configured to guide the user to complete a corresponding recipe step.

It should be noted that, because for the implementations and beneficial effects of the foregoing modules, reference may be made to the detailed description of the corresponding steps in the foregoing embodiments, the details are not described in this embodiment.

As can be understood that in the technical solution in this embodiment, only one recipe step of the target recipe is displayed on one presenting page, which facilitate a user to quickly locate a certain recipe step. Less content is shown on a single presenting page, which facilitates the user to quickly view operation content of a certain recipe step. Meanwhile, because the presenting page of each recipe step is configured with an interactive tool capable of being invoked by the user, and the interactive tool can guide the user to complete a corresponding recipe step, compared with a method in the prior art which requires manual and repeated memorizing or repeated viewing of a certain recipe step to complete the cooking, the technical solution in this embodiment greatly improves cooking efficiency and user experience.

It should be noted that the technical solution in this embodiment may be presented in the form of a user APP in specific practice, and the user APP needs to be loaded and run in an electronic device. The electronic device includes: a server-side electronic device and a user-side electronic device, including but not limited to: a mobile phone, a computer, a notebook computer, a tablet computer, an intelligent wearable device, or the like. The tablet computer may be a household handheld tablet computer, or a tablet computer embedded in a kitchen device, for example, a tablet computer embedded in a range hood, a tablet computer embedded in an oven, or a tablet computer embedded in a refrigerator.

### Embodiment VII

An electronic device shown according to an exemplary embodiment is provided. The electronic device includes:

a wireless communication module, a processor, and a memory, wherein program instructions are stored in the memory; and

the processor is configured to execute the program instructions stored in the memory, and perform the method for display an electronic recipe according to Embodiment V.

In the electronic device in this embodiment, for the specific implementation form of the wireless communication module, the processor and the memory, reference may be made to Embodiment III. Details are not described herein.

As can be understood that in the technical solution in this embodiment, only one recipe step of the target recipe is displayed on one presenting page, which facilitate a user to quickly locate a certain recipe step. Less content is shown on a single presenting page, which facilitates the user to quickly view operation content of a certain recipe step. Meanwhile, because the presenting page of each recipe step is configured with an interactive tool capable of being invoked by the user, and the interactive tool can guide the user to complete a corresponding recipe step, compared with a method in the prior art which requires manual and repeated memorizing or repeated viewing of a certain recipe step to complete the cooking, the technical solution in this embodiment greatly improves cooking efficiency and user experience.

### Embodiment VIII

A computer-readable storage medium shown according to an exemplary embodiment is provided, where the computer-readable storage medium stores an erasable computer program; and

when the computer program runs on a computer device, the computer device is enabled to perform the method for display an electronic recipe according to Embodiment V.

For the specific implementation form of the computer-readable storage medium according to this embodiment, reference may be made to Embodiment IV. Details are not described herein.

As can be understood that in the technical solution in this embodiment, only one recipe step of the target recipe is displayed on one presenting page, which facilitate a user to quickly locate a certain recipe step. Less content is shown on a single presenting page, which facilitates the user to quickly view operation content of a certain recipe step. Meanwhile, because the presenting page of each recipe step is configured with an interactive tool capable of being invoked by the user, and the interactive tool can guide the user to complete a corresponding recipe step, compared with a method in the prior art which requires manual and repeated memorizing or repeated viewing of a certain recipe step to complete the cooking, the technical solution in this embodiment greatly improves cooking efficiency and user experience.

As can be understood that for the same or similar parts in the foregoing embodiments, reference may be made to each other, and for the content not described in detail in some embodiments, reference may be made to the same or similar content in other embodiments.

It should be noted that in the description of the application, the terms “first”, “second”, and the like are merely only used for descriptive purposes and cannot be understood as indicating or implying relative importance. In addition, in the description of the application, unless otherwise specified, “a plurality of” means at least two.

Any process or method description in the flowchart or otherwise described herein may be understood to represent a module, a fragment, or a portion of code including one or more executable instructions for implementing steps of a particular logical function or process, and the scope of the preferred implementation of the application includes additional implementations, wherein the functions may not be performed in the order shown or discussed, including the order according to the functions involved in a substantially simultaneous manner or in a reverse order, as should be understood by a person skilled in the art to which the embodiments of the application belong.

It should be understood that each part of the application may be implemented by hardware, software, firmware or a combination thereof. In the foregoing embodiments, a plurality of steps or methods may be implemented by software or firmware stored in a memory and executed by a suitable instruction execution system. For example, if the steps or methods are implemented by hardware, same as in another embodiment, the steps or methods may be implemented by any one or a combination of the following technologies known in the art: a discrete logic circuit with a logic gate circuit for implementing a logical function on a data signal, an application-specific integrated circuit with a suitable combinational logic gate circuit, a programmable gate array (PGA), a field programmable gate array (FPGA), and the like.

A person of ordinary skill in the art may understand that all or some steps in the methods of the foregoing embodiments may be implemented by instructing relevant hardware through a program. The program may be stored in a computer-readable storage medium, and when the program is executed, one of the steps of the method embodiment or a combination thereof is included.

In addition, functional units in each embodiment of the application may be integrated into one processing module, or each of the units may exist alone physically, or two or more units may be integrated into one module. The integrated modules may be implemented in a form of hardware, or may be implemented in a form of a software function module. The integrated module may alternatively be stored in one computer-readable storage medium if implemented in the form of a software function module and sold or used as a standalone product.

The foregoing storage medium may be a read-only memory, a magnetic disk, an optical disc, or the like.

In the description of this specification, the description with reference to the terms “one embodiment”, “some embodiments”, “examples”, “specific examples”, “some examples”, or the like means that specific features, structures, materials or characteristics described in combination with the embodiments or examples are included in at least one embodiment or example of the application. In the description, the illustrative expressions of the foregoing terms do not necessarily refer to the same embodiments or examples. In addition, the specific features, structures, materials, or characteristics described may be combined in a suitable manner in any one or more embodiments or examples.

Although the embodiments of the application have been shown and described above, it can be understood that the foregoing embodiments are examples and cannot be construed as limiting the application, and a person of ordinary skill in the art may make changes, modifications, replacements and variations to the foregoing embodiments within the scope of the application.

What is claimed is:

1. A method for processing an electronic recipe applied to an electronic device, comprising:
obtaining a source file of a target recipe, the source file comprising a plurality of recipe steps of the target recipe; and
creating one presenting page for each recipe step of the target recipe, wherein
the presenting page of each recipe step is configured with an interactive tool capable of being invoked by a user, and the interactive tool is configured to guide the user to complete a corresponding recipe step.

2. The method according to claim 1, wherein the interactive tool comprises at least one of the following:
a timer, a speech recognizer, a gesture recognizer, and a speech announcer.

3. The method according to claim 2, wherein
under a condition that the interactive tool comprises a timer, and any one recipe step is configured with timing, the presenting page of the recipe step is configured to start timing based on a user-triggered operation, and display countdown duration;
under a condition that the interactive tool comprises a speech recognizer, the presenting page of the recipe step is configured to turn a page based on the user's page turning speech instruction;
under a condition that the interactive tool comprises a gesture recognizer, the presenting page of the recipe step is configured to turn a page based on page turning gesture instruction;
under a condition that the interactive tool comprises a gesture recognizer, the presenting page of the recipe step is further configured to display a page turning gesture prompt icon under a condition that no user operation is detected within a preset duration; and
under a condition that the interactive tool comprises a speech announcer, the presenting page of the recipe step is configured to start speech broadcast of the recipe step based on a user instruction, and display a working state of the speech announcer.

4. The method according to claim 1, further comprising:
under a condition that all recipe steps in the source file correspond to one parent file, prompting the user to specify a recipe step cutting node when uploading the source file, cutting the parent file into a plurality of subfiles based on the cutting node specified, wherein each subfile corresponds to a recipe step, and prompting the user to add operation content and a food ingredient description of each recipe step; and
under a condition that each recipe step in the source file corresponds to a subfile, prompting the user to add operation content and a food ingredient description of each recipe step when uploading the source file.

5. The method according to claim 4, wherein the presenting page of each recipe step is further configured to:
display the operation content, the food ingredient description and nutritional ingredient content calculated based on the food ingredient description of each recipe step, wherein the food ingredient description comprising food ingredient types and food ingredient weights; and
under a condition that food ingredient types and/or food ingredient weights on any presenting page are modified by the user, calculate modified nutritional ingredient content in real time, and present a calculation result to the user.

6. The method according to claim 1, further comprising:
associating at least one cooking device with the target recipe, and establishing a list of cooking devices according to the at least one cooking device, wherein each cooking device can be used to cook food following the target recipe;
creating a detail page for the target recipe, wherein the detail page comprises recipe information corresponding to a first cooking device selected by the user from the list and an icon of each cooking device in the list.

7. The method according to claim 6, further comprising:
acquiring recipe information of a second cooking device, wherein the second cooking device is other cooking devices except the first cooking device in the list;
creating a switching page of a detail page of a target recipe according to the recipe information of the second cooking device, the switching page being entered by clicking on an icon of the second cooking device.

8. The method according to claim 6, wherein
the detail page of the target recipe is further configured with a first touch button for controlling the first cooking device to start cooking and a link associated with the presenting page of a first recipe step of the target recipe, wherein the presenting page of a first recipe step can be switched to other presenting page of the recipe steps in order.

9. The method according to claim 7, wherein
the switching page is further configured with a second touch button for controlling the second cooking device to start cooking and a link associated with a presenting page of a first recipe step of the target recipe, wherein the presenting page of a first recipe step can be switched to other presenting page of the recipe steps in order.

10. The method according to claim 6, further comprising:
creating a smart cooking parameter setup page which is configured to provide the user with options for food ingredient types and food ingredient weights, and recommend an optimum cooking temperature and cooking duration based on food ingredient types and food ingredient weights selected by the user.

11. A method for processing an electronic recipe applied to an electronic device, comprising:
obtaining a source file of a target recipe, the source file comprising a plurality of recipe steps of the target recipe;
displaying one recipe step of the target recipe on a presenting page;
the presenting page is configured with an interactive tool capable of being invoked by a user, and the interactive tool is configured to guide the user to complete a corresponding recipe step.

12. The method according to claim 11, wherein the interactive tool comprises at least one of the following:
a timer, a speech recognizer, a gesture recognizer, and a speech announcer.

13. The method according to claim 12, further comprising:
under a condition that the interactive tool comprises a timer and the timer has been set for each recipe step when the source file of the target recipe is uploaded to a server-side electronic device, starting timing based on a user-triggered operation when the recipe step is displayed, and displaying countdown duration on the presenting page of the recipe step;
under a condition that the interactive tool comprises a speech recognizer, the presenting page of the recipe step is configured to turn a page based on the user's page turning speech instruction;
under a condition that the interactive tool comprises a gesture recognizer, the presenting page of the recipe step is configured to turn a page based on page turning gesture instruction;
under a condition that the interactive tool comprises a gesture recognizer, the presenting page of the recipe step is further configured to display a page turning gesture prompt icon under a condition that no user operation is detected within a preset duration; and
under a condition that the interactive tool comprises a speech announcer, on the presenting page of the recipe step is configured to start speech broadcast of the recipe step based on a user instruction, and display a working state of the speech announcer.

14. The method according to claim 11, wherein the presenting page of each recipe step is configured to:
display the operation content, the food ingredient description and nutritional ingredient content calculated based on the food ingredient description of each recipe step, wherein the food ingredient description comprising food ingredient types and food ingredient weights; and
under a condition that food ingredient types and/or food ingredient weights on any presenting page are modified by the user, calculate modified nutritional ingredient content in real time, and present a calculation result to the user.

15. The method according to claim 11, further comprising:
obtaining a list of cooking devices associated with the target recipe, wherein the list of cooking devices comprises at least one cooking device, and each cooking device can be used to cook food following the target recipe;
displaying a detail page for the target recipe, wherein the detail page comprises recipe information corresponding to a first cooking device selected by the user from at least one cooking device and an icon of each cooking device.

16. The method according to claim 15, further comprising:
under a condition that the user clicks on an icon of a second cooking device, wherein the second cooking device is other cooking devices except a first cooking device in the list, displaying a recipe information of the second cooking device instead of recipe information of the first cooking device on the detail page for the target recipe.

17. The method according to claim 15, further comprising: after all presenting pages of the recipe steps, displaying a smart cooking parameter setup page which is configured to provide the user with options for food ingredient types and food ingredient weights, and recommend an optimum cooking temperature and cooking duration based on food ingredient types and food ingredient weights selected by the user.

18. An electronic device, comprising a processor and a memory, wherein program instructions are stored in the memory; and
when the program instructions are executed by the processor, the processor is enabled to perform:
obtaining a source file of a target recipe, the source file comprising a plurality of recipe steps of the target recipe; and
creating one presenting page for each recipe step of the target recipe, wherein
the presenting page of each recipe step is configured with an interactive tool capable of being invoked by a user, and the interactive tool is configured to guide the user to complete a corresponding recipe step.
